(19) United States
(12) Patent Application Publication
(10) Pub. No.: US 2022/0304971 A1
(43) Pub. Date: Sep. 29, 2022

(54) BEVERAGE FORTIFIED WITH FERROUS BISGLYCINATE

(71) Applicant: SOCIETE DES PRODUITS NESTLE S.A., Vevey (CH)

(72) Inventors: EDWIN ALBERTO HABEYCH NARVAEZ, Lausanne (CH); LAURENCE SANDOZ, Echallens (CH); JOYDEEP RAY, Epalinges (CH); NICOLA GALAFFU, Ornex (FR); CATHERINE MACE, La Conversion (CH)

(21) Appl. No.: 17/596,945

(22) PCT Filed: Jun. 25, 2020
(86) PCT No.: PCT/EP2020/067944

§ 371 Date: Dec. 22, 2021

(30) Foreign Application Priority Data
Jun. 25, 2019 (EP) 19182384.8

(51) Int. Cl. A61K 31/295; A23L 2/52; A23L 2/02; A23L 33/165; A23L 33/105; A23L 33/155; A23L 33/00; A61K 9/00; A61K 33/06; A61K 47/46; A61K 33/30; A61K 33/18; A61K 31/593; A61K 31/714

(52) U.S. Cl. CPC A61K 31/295; A23L 2/52; A23L 2/02; A23L 33/165; A23L 33/105; A23L 33/155; A23L 33/40; A61K 9/0095; A61K 33/06; A61K 47/46; A61K 33/30; A61K 33/18; A61K 31/593; A61K 31/714; A23V 2002/00

(57) ABSTRACT

The present invention relates generally to the field of beverages. In particular, the present invention relates to the field of beverages fortified with iron. For example, the present inventors have found that iron fortification with ferrous bisglycinate in a plant based beverage with a pH in the range of 3.5 to 3.9 leads to an increased color stability and a better taste of the beverage compared to other compounds commonly used to fortify beverages with iron.

### TECHNICAL FIELD

The present invention relates generally to the field of beverages. In particular, the present invention relates to the field of beverages fortified with iron. For example, the present inventors have found that iron fortification with ferrous bisglycinate in a plant based beverage with a pH in the range of 3.5 to 3.9 leads to an increased color stability and a better taste of the beverage compared to other compounds commonly used to fortify beverages with iron.

### BACKGROUND OF THE INVENTION

The number of people enjoying a vegan, vegetarian or flexitarian diet is increasing significantly in recent years.

The Academy of Nutrition and Dietetics states in J Acad Nutr Diet. 2016 Dec.;116(12):1970-1980, that appropriately planned vegetarian, including vegan, diets are healthful, nutritionally adequate, and may provide health benefits for the prevention and treatment of certain diseases; and that these diets are appropriate for all stages of the life cycle, including pregnancy, lactation, infancy, childhood, adolescence, older adulthood, and for athletes. However, the article states as well that in particular vegans need reliable sources of vitamin B12.

Hence, while a diet omitting meat or even all animal products can be healthy, there is a certain risk that micronutrients that are naturally present in animal products are not consumed in sufficient quantities. For example, sufficient amounts of vitamin B12, calcium, iron, and zinc need to be consumed. Omitting meat or even all animal products might lead to deficiencies and related disorders, in particular later in life. While it is possible to plan a meatless diet, or even a diet free from animal products, that contains all the required micro-and macro-nutrients, this requires expertise, care and discipline.

The amount of available time, nutritional knowledge, nutritional incompatibilities, and/or food preferences, for example, may occasionally prevent vegans, vegetarians or flexitarians from consuming a diet that contains all the required micronutrients in sufficient amounts.

Available today are nutritional supplements, usually in capsule or tablet form, that help to address some of the possible nutritional deficiencies that may arise from consuming no meat or no animal products.

However, such products have the disadvantage that they have an unnatural appearance and due to their format allow easy overdosing, for example.

A very important micronutrient that needs to be consumed in sufficient amounts and that typically is present in meat or fish, but also in certain vegetables, is iron. An adequate intake of iron may help prevent iron deficiency, and/or reduce the risk of iron deficiency. Iron deficiency is one of the most common causes of anaemia which may result in cognitive impairment, decreased physical capacity and reduced immunity. Iron deficiency also contributes to diminished growth and learning.

Plant based beverages are perceived much more natural than nutritional supplements in capsule or tablet format. Also, typically, they have a very pleasant taste, making it easy to consume them regularly.

However, iron fortification in plant based beverages is often a problem, as the iron fortification typically leads to an alteration in colour over storage time. Typical iron fortifying agents used in the state of the art, such as iron sulfate, NaFeEDTA or iron pyrophosphate, still lead to variation in colour during storage time. Also, occasionally, iron sulfate, NaFeEDTA or iron pyrophosphate are not perceived as natural by consumers.

WO 2005/006871 A1 discloses a water beverages containing tea extract, including tea catechins, and polyvalent mineral cations, such as calcium. The water beverages exhibit a relatively low turbidity.

Miglioranza et al, Nutrition 19(5):419-421, 2003, investigates the effect of long-term fortification of whey drink with ferrous bisglycinate on anaemia prevalence in children and adolescents from deprived areas in Londrina (Paraná, Brazil).

Any reference to prior art documents in this specification is not to be considered an admission that such prior art is widely known or forms part of the common general knowledge in the field.

### SUMMARY OF THE INVENTION

Accordingly, there is a need to at least partially overcome a disadvantage of the present state of the art while overcoming the potential nutritional deficits possibly arising from a diet omitting meat and/or animal products.

Hence, it was the objective of the present invention to enrich or improve the state of the art, and, in particular, to provide a composition with a great taste that can be used to overcome the potential nutritional deficits possibly arising from a diet omitting meat and/or animal products, that is perceived more natural than nutritional supplements available today and/or that allows iron fortification with as little colour change during storage times as possible and/or that allows to minimize a negative impact on taste; or at least to provide a useful alternative.

The inventors were surprised to see that the objective of the present invention could be achieved by the subject matter of the independent claims. The dependent claims further develop the idea of the present invention.

Accordingly, the present invention provides a fortified plant based composition, wherein said fortified plant based composition has a pH in the range of 3.5-3.9 and is fortified with ferrous bisglycinate.

The present inventors were surprised to find that iron fortification in a plant based composition at a pH in the range of 3.5-3.9 with ferrous bisglycinate leads to less colour change in 8 week shelf life tests than iron fortification with other commonly used iron sources.

Moreover, the inventors also found that fortification with ferrous bisglycinate led to a less metallic taste that is otherwise frequently observed when fortifying plant based compositions with iron. Even further, consumers perceive ferrous bisglycinate as more natural than iron sulfate, NaFeEDTA or iron phosphate. Moreover, in terms of absorption, ferrous bisglycinate is less affected by polyphenols present in the fruits as this compound is chelated. The fortified plant based composition may be a beverage. For example, the fortified plant based composition may comprise components from apple, mango, lime, and turmeric. The inventors find that this combination of ingredients addresses the nutritional requirements of vegans, vegetarians and flexitarians very well. However, the resulting colour of the beverage is yellow. Here, in particular, it is critical that a change in colour due to iron fortification is minimized as far as possible, as otherwise the resulting fortified beverage would turn brown relatively quickly, making it unappealing for consumers.

As used in this specification, the words “comprises”, “comprising”, and similar words, are not to be interpreted in an exclusive or exhaustive sense. In other words, they are intended to mean “including, but not limited to”.

The present invention further provides a fortified plant based composition for use in the prevention or treatment of disorders linked to malnutrition, in particular disorders linked to malnutrition arising from omitting some or all animal products in the diet.

Even further, the present invention provides the use of ferrous bisglycinate to fortify a plant based composition at a pH in the range of 3.5-3.9 with iron, while reducing unwanted metallic taste and/or a colour change in the composition.

### BRIEF DESCRIPTION OF THE DRAWING

FIG. 1 shows the results of example 2 below.

### DETAILED DESCRIPTION OF THE INVENTION

Consequently, the present invention relates in part to a fortified plant based composition, wherein said fortified plant based composition is fortified with ferrous bisglycinate; said fortified fruit based composition comprises fruit components from apple, mango, lime, and turmeric,; said fortified plant based composition is a beverage; and said fortified plant based composition has a pH in the range of 3.5-3.9.

For the purpose of the present invention the term “fortified” shall mean that nutrients are added to food compositions at higher levels than what the original food provides.

In accordance with Codex Alimentarius, the term “food” shall mean for the purpose of the present invention any substance, whether processed, semi-processed or raw, which is intended for human consumption, and includes drink, chewing gum and any substance which has been used in the manufacture, preparation or treatment of “food” but does not include cosmetics or tobacco or substances used only as drugs.

The term “plant based” shall be understood for the purpose of the present invention to comprise parts of plants that are consumed by humans or other animals as food, for example fruits and/or vegetables. For the purpose of the present invention, a composition shall be considered plant based, if it contains at least 50 weight-% plant material.

Ferrous bisglycinate can be obtained commercially from many chemical vendors. Studies have shown, that ferrous bisglycinate has a better bioavailability compared to other compounds used for iron fortification (J Nutr 2002;130;2195-2199).

The inventors have found that a composition comprising components from apple, mango, lime, and turmeric meets the nutritional requirements of people on a vegan, vegetarian or flexitarian diet particularly well. The resulting yellow colour of the composition requires to ensure that the iron fortification leads to an as little change in colour as possible during storage time to avoid that the colour of the composition turns brown.

For the purpose of the present invention, the term “vegan” diet shall mean a diet abstaining from the use of animal products. For the purpose of the present invention, the term “vegetarian” diet shall mean a diet abstaining from the consumption of meat or fish. For the purpose of the present invention, the term “flexitarian” diet shall mean a normal diet abstaining in regular intervals from meat or fish.

The plant based composition of the present invention is a beverage. Beverages have the advantage that they can be easily consumed without the need of utensils such as a spoon, for example, and are, hence, effortless to consume, both, at home and “on the go”.

The inventors have found that if in the plant based composition of the present invention ferrous bisglycinate is used for iron fortification, alterations in colour during storage are less than with other iron fortifying compounds, a metallic off-taste is largely avoided and a high bioavailability of the iron after consumption is ensured. These positive effects are obtained particularly well, if the pH of the plant based composition is in the range of 3.5-3.9.

The perceived acidity in the composition is an essential element of its taste. If the acidity is too low, the composition will lack freshness. On the other hand, too much acidity may give the composition a too sharp taste and makes it unpleasant to drink. It is hence important to seek the right equilibrium. Hence, the plant based composition in accordance with the present invention may have an acidity in the range of 0.65-0.85 g/100g, expressed as Citric Acid Anhydrous at pH 8.1 according to International Fruit and Vegetable Juice Association (IFU) method no. 3, Rev. 2017. For example, the plant based composition in accordance with the present invention may have an acidity in the range of 0.72-0.77 g/100g, calculated at CAA at a pH of 8.1.

The fortified plant based composition of the present invention may comprise components from any part of the plant that is considered edible. For example, these parts of the plants maybe fruit or vegetables, but could also be leaves or roots, for example. The components may be selected from the group consisting of juice or puree. In one embodiment of the present invention the juice and/or puree is obtained from fruit and/or vegetables.

Further ingredients may be added to the composition of the present invention to further improve the taste experience and/or to make the nutrient profile of the resulting composition even more suitable for the nutritional needs of people avoiding animal products in their diet partially or completely. As such, the composition of the present invention may further comprise pepper puree, ginger juice and/or chili powder.

For example, the fortified plant based composition according to the present invention may comprise apple juice in the range of 40-60 weight-%, mango puree in the range of 30-50 weight-%, pepper puree in the range of 1-2 weight-%, lime juice in the range of 5-8 weight- %, ginger juice in the range of 1-2 weight-%, turmeric juice in the range of 0.1-1 weight-%, lemon juice in the range of 0.01-0.1 weight-% and chili powder in the range of 0.01-0.1 weight-%.

Further, for example, the fortified plant based composition according to the present invention may comprise apple juice in the range of 45-55 weight-%, mango puree in the range of 35-45 weight-%, pepper puree in the range of 1.1-1.4 weight-%, lime juice in the range of 5-7 weight-%, ginger juice in the range of 1.1-1.3 weight-%, turmeric juice in the range of 0.05-0.09 weight-%, lemon juice in the range of 0.05-0.09 weight-% and chili powder in the range of 0.01-0.05 weight-%.

An embodiment of the present invention may comprise about 49,41 weight- % apple juice, about 40,0 weight-% mango puree, about 6,4 weight-% lime juice, about 1,3 weight-% pepper puree, about 1,2 weight-% ginger juice, about 0,7 weight-% turmeric juice, about 0,07 weight-% lemon juice, and about 0,02 weight-% chili powder.

Calcium is needed, for example, for strong and healthy bones and teeth. People on a normal diet obtain calcium for example from dairy foods, such as milk, cheese, or yoghurt, for example. Of course there are also plant-based foods that contain calcium, such as cabbage and pulses, for example, but sometimes they are not consumed in sufficient amounts. Hence, the plant-based composition of the present invention may also be fortified with a calcium source. For example, the fortified plant based composition according to the present invention may further comprise calcium lactate. Calcium lactate is commercially available from many sources. Calcium may be added to the composition of the present invention, so that the composition of the present invention comprises calcium in an amount in the range 10-800 mg/100ml of the composition, 50-500 mg/100m1 of the composition, 80-300 mg/100m1 of the composition, 100-200 mg/100m1 of the composition, or 110-130 mg/100m1 of the composition. For example, calcium may be added to the composition of the present invention, so that the composition of the present invention comprises calcium in an amount of 120 mg/100m1 of the composition.

For example, the fortified plant based composition according to the present invention may comprise calcium lactate in the range of 0.5-1.5 weight-%, for example 0.8-1.1 weight-%. In one embodiment of the present invention the fortified plant based composition may comprise about 0.9 weight-% calcium lactate.

Consumers value, if a composition, even if it is fortified, contains as much plant-based material as possible. Hence, the fortified plant based composition of the present invention may have a plant content of at least 75 weight-%, at least 80 weight-%, at least 90 weight-% or at least 95 weight-%. For example, the fortified plant based composition of the present invention may have a plant content of at least 99 weight-%.

The plant based composition of the present invention may comprise ingredients that are sourced from organic agriculture. In one embodiment of the present invention, the composition fulfils all the requirements to be labelled “organic”.

To even better meet the nutritional requirements of people avoiding animal products in their diet partially or completely, the composition of the present invention may be further fortified. For example, the fortified plant based composition may be further fortified with nutrients, vitamins and/or minerals.

It is within the skill of a skilled artesian to appropriately select nutrients, vitamins and/or minerals accordingly. For example, nutrients, vitamins and/or minerals may be selected from the group consisting of zinc sulfate, calcium lactate, potassium iodide, vitamin

D3, vitamin B12 and any mixture of any of the foregoing. The fortified plant based composition of the present invention may be also further fortified with Zinc, Vitamin B12 and/or Vitamin D. Vitamin D, for example vitamin D3, may be vegan vitamin D or vegan vitamin D3, respectively.

In one embodiment of the present invention, the fortified plant based composition of the present invention is fortified with zinc sulfate, calcium lactate, potassium iodide, vitamin D, and vitamin B12. This has the advantage that the composition of the present invention is very well fortified to address deficiencies that might arise from a vegan, vegetarian or flexitarian diet.

Zinc is an important micronutrient that plays, e.g., a role in immunity and is available from many animal sources. It is also available from plant sources, such as tomatoes, seeds, lentils and beans, for example. However, since plant sources of zinc often contain inhibitors of zinc absorption, such as phytate, for example, there is a risk of zinc deficiency. Consequently, fortification with zinc may be advisable. Zinc can be provided, for example in the form of zinc sulfate, which is commercially available.

The body needs iodine, e.g., to make thyroid hormones. Iodine is present in fish, eggs or dairy products, for example. Vegan iodine sources include for example seaweed, however, sufficient iodine supply needs to be ensured. Iodine can be provided, for example in the form of potassium iodide, which is commercially available.

Vitamin D is present, e.g., in fatty fish, beef liver, or cheese. It is involved in the regulation of blood calcium and phosphorus levels and thus plays an important role in bone health. Plant-based vitamin D is available from mushrooms, for example. If people do not spend sufficient time outside, there is a risk of vitamin D deficiency. Consequently, fortification with vitamin D may be advisable. Vitamin D is commercially available. Vitamin D3, or cholecalciferol, is a vitamin D and plays a role in the regulation of healthy bones, muscle, immune system, insulin and metabolism. It is generally believed to be more potent than vitamin D2. Consequently, fortification with vitamin D3 may be advisable. Vitamin D3 is commercially available.

Vitamin B12 is important in the normal functioning of the nervous system due to its role in the synthesis of myelin, and in the maturation of developing red blood cells in the bone marrow. It is naturally found in animal products such as fish, meat, poultry, eggs, milk, and milk products. However, vitamin B12 is generally not present in plant-based foods, unless they were fortified with vitamin B12. Consequently, fortification with vitamin B12 may be advisable. Vitamin B12 is commercially available.

The composition of the present invention may be designed so that it is specifically suitable for the nutritional requirements of a flexitarian, a vegetarian or a vegan. Hence, in one embodiment of the present invention the fortified plant based composition is suitable for consumption by a vegan and/or to be consumed by a vegan. In another embodiment of the present invention the fortified plant based composition is suitable for consumption by a vegetarian and/or to be consumed by a vegetarian. In a further embodiment of the present invention the fortified plant based composition is suitable for consumption by a flexitarian and/or to be consumed by a flexitarian.

Consequently, the plant based composition according to the present invention may comprise iron in an amount of 1.5-9 mg/100 g of the composition, zinc in an amount of 1-8 mg/100 g of the composition, vitamin B12 in an amount of 2-7 μg/100 g of the composition, and vitamin D in an amount of 3-20 μg/100 g of the composition.

For example, the plant based composition according to the present invention may comprise iron in an amount of 1.5-5 mg/100 g of the composition, zinc in an amount of 1-4 mg/100 g of the composition, vitamin B12 in an amount of 2-7 μg/100 g of the composition, and vitamin D in an amount of 3-10 μg/100 g of the composition.

Further, for example, the plant based composition according to the present invention may comprise iron in an amount of 2-3 mg/100 g of the composition, zinc in an amount of 1-2 mg/100 g of the composition, vitamin B12 in an amount of 2-3 μg/100 g of the composition, and vitamin D in an amount of 4-6 μg/100 g of the composition.

Further, for example, the plant based composition according to the present invention may comprise iron in an amount of about 2.1 mg/100 g of the composition, zinc in an amount of about 1.5 mg/100 g of the composition, vitamin B12 in an amount of about 2.5 μg/100 g of the composition, and vitamin D in an amount of about 5 μg/100 g of the composition.

The fortified plant based in accordance with the present invention may be for use in the prevention or treatment of disorders linked to malnutrition, in particular disorders linked to malnutrition arising from omitting some or all animal products in the diet.

Such disorders linked to malnutrition arising from omitting some or all animal products in the diet are well-known to the skilled artesian. For example, they may be selected from the group consisting of anaemia, goitre, risk of osteomalacia and osteoporosis, impaired growth velocity, and neurological problems, or combinations thereof.

The term “treat” or “treatment” as used herein encompasses amelioration and/or alleviation of a disorder i.e. the amelioration and/or alleviation of the symptoms of a disorder or conditions associated with such disorders. It may for example encompass the reduction of the severity of a disorder in a subject.

The term “prevent” or “prevention” as used herein refers to the prevention of the occurrence, or reduction of the risk of the occurrence, of a disorder or conditions associated with such disorders in a subject.

The term “subject” as used herein refers to a mammal and more particularly a cat, a dog or a human. The human may be an adult, child or infant. In an embodiment of the invention the subject is a mammal selected from the group consisting of a cat, a dog and, a human.

The subject matter of the present invention further extends to the use of ferrous bisglycinate to fortify a plant based composition at a pH in the range of 3.5-3.9 with iron while reducing unwanted metallic taste and/or a colour change in the composition.

The plant based composition may be any composition in accordance with the present invention described above.

For example, the plant based composition may be a plant based composition, wherein said plant based composition comprises components from apple, mango, lime, and turmeric.

The plant based composition may be fortified and/or may be a beverage.

Those skilled in the art will understand that they can freely combine all features of the present invention disclosed herein. In particular, features described for the product of the present invention may be combined with the use of the present invention and vice versa. Further, features described for different embodiments of the present invention may be combined.

Although the invention has been described by way of example, it should be appreciated that variations and modifications may be made without departing from the scope of the invention as defined in the claims.

The term “and/or” used in the context of the “X and/or Y” should be interpreted as “X”, or “Y”, or “X and Y”.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 8, from 3 to 7, from 4 to 9, from 3.6 to 4.6, from 3.5 to 9.9, and so forth.

All references to singular characteristics or limitations of the present invention shall include the corresponding plural characteristic or limitation, and vice versa, unless otherwise specified or clearly implied to the contrary by the context in which the reference is made.

Unless defined otherwise, all technical and scientific terms used herein have the same meaning as commonly understood by one of ordinary skill in the art.

Furthermore, where known equivalents exist to specific features, such equivalents are incorporated as if specifically referred in this specification. Further advantages and features of the present invention are apparent from the figures and non-limiting examples.

### EXAMPLES

Example 1:

A composition in accordance with the present invention was prepared with the following ingredients: 49.41% apple juice, 40.0% mango puree, 6.4% lime juice, 1.3% pepper puree, 1.2% ginger juice, 0.9% calcium lactate, 0.7% turmeric juice, 0.07% lemon juice, 0.02% chili powder. “%” refers to “weight-%”.

This composition was adjusted to a pH or 3.8 and fortified by mixing 2.1 mg of iron from different sources/100 g product, which correspond to about 15% of Recommended Daily allowance for adults. Subsequently, 80 grams aliquots of the fortified samples were filled in 125 mL glass bottles (Schott Duran) and pasteurized at 90° C. for 1 min. A non-fortified control sample was prepared in a similar manner as described above and used as a reference.

In order to quantify the changes in color produced as consequence of the presence of iron in the yellow recipe, color analysis was carried out using a reflectance spectrometer (X-Rite ColorEye 7000A) set to a D65 illuminant and a 10° observer. Values of a* (the amount of red and green), b* (the amount of yellow and blue), L* (the amount of luminosity from black to white) were recorded in triplicate from three independent samples obtained for each treatment at different time points. The total color deviation (ΔE) of each sample was calculated according to the following equation.

$$Δ E = \sqrt{{( {L}_{c o n trol}^{*} - {L}_{sample}^{*} )}^{2} + {( {a}_{control}^{*} - {a}_{sample}^{*} )}^{2} + {( {b}_{control}^{*} - {b}_{sample}^{*} )}^{2}}$$

ΔE refers to a measure of the overall color change in the sample.

The results shown in table 1 indicate that iron fortification with iron bisglycinate in this beverage matrix and at a pH in the range of 3.5 to 4.0 surprisingly outperforms other state of the art compounds that are used to fortify compositions with iron, for example in terms of color stability.

TABLE 1
Results of example 1-Beverage sample @ 37° C.

| | ΔE ± SD | | | |
|---|---|---|---|---|
| Time (weeks) | FeSO₄ | NaFeEDTA | FePP | Fe bisglycinate |
| 0 | 10.59 ± 0.03 | 10.99 ± 0.14 | 12.80 ± 0.07 | 10.13 ± 0.05 |
| 2 | 11.93 ± 0.16 | 12.85 ± 0.06 | 13.84 ± 0.31 | 11.91 ± 0.10 |
| 5 | 14.33 ± 0.07 | 14.46 ± 0.14 | 17.22 ± 0.04 | 12.37 ± 0.06 |

Example 2:

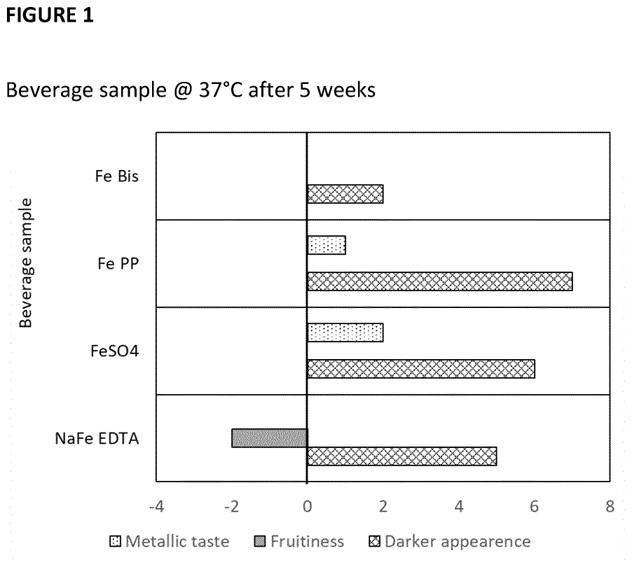

The inventors have also tasted the product composition prepared and incubated at 37° C. for 5 weeks in order to simulate accelerated shelf life condition. FIG. 1 demonstrates the difference of the iron fortified beverage samples for different sensory attributes. The inventors have scored each attributes according to the intensity perceived for each iron fortified beverage samples compared to a non-fortified fresh sample. The scores for each attributes from different inventors were combined and plotted. It was surprising to see that iron fortification with ferrous bisglycinate upon incubation leads to lower colour change, perceived noticeably less metallic in taste in the beverage sample compared to the other tested compounds used to fortify beverages with iron.

1. A fortified plant based composition, wherein
said fortified plant based composition is fortified with ferrous bisglycinate,
said fortified plant based composition comprises components selected from the group consisting of apple, mango, lime, and turmeric,
said fortified plant based composition is a beverage, and
said fortified plant based composition has a pH in the range of 3.5-3.9.

2. A fortified plant based composition according to claim 1, wherein the fortified plant based composition has an acidity in the range of 0.65-0.85 g/100g, calculated at CAA at a pH of 8.1.

3. A fortified plant based composition according to claim 1 wherein the components are selected from the group consisting of juice or puree.

4. A fortified plant based composition according to claim 1, further comprising pepper puree, ginger juice and/or chili powder.

5. A fortified plant based composition according to claim 1, further comprising calcium lactate.

6. A fortified plant based composition according to claim 1, wherein the fortified plant based composition has a fruit content of at least 75 weight-%.

7. A fortified plant based composition according to claim 1, wherein the fortified plant based composition is further fortified with nutrients, vitamins and minerals.

8. A fortified plant based composition according to claim 7, wherein the nutrients, vitamins and minerals are selected from the group consisting of zinc sulfate, calcium lactate, potassium Iodide, vitamin D3, vitamin B12 and any mixture of any of the foregoing.

9. A fortified plant based composition according to claim 7, wherein fortified plant based composition is further fortified with Zinc, Vitamin B12 and Vitamin D.

10. A fortified plant based composition according to claim 1, wherein the fortified plant based composition is suitable for consumption by a vegan.

11. A fortified plant based composition according to claim 1, wherein the fortified plant based composition comprises apple juice in the range of 40-60 weight-%, mango puree in the range of 30-50 weight-%, pepper puree in the range of 1-2 weight-%, lime juice in the range of 5-8 weight-%, ginger juice in the range of 1-2 weight-%, turmeric juice in the range of 0.1-1 weight-%, lemon juice in the range of 0.01-0.1 weight-% and chili powder in the range of 0.01-0.1 weight-%.

12. A fortified plant based composition according to claim 1, wherein the fortified plant based composition comprises calcium lactate in the range of 0.5-1.5 weight-%.

13. A fortified plant based composition according to claim 1, wherein the fortified plant based composition comprises iron in an amount of 1.5-9 mg/100 g of the composition, zinc in an amount of 1-8 mg/100 g of the composition, vitamin B12 in an amount of 2-7 μg/100 g of the composition, and vitamin D in an amount of 3-20 μg/100 g of the composition.

14. A method for the prevention or treatment of disorders linked to malnutrition comprising adminstering to an individual in need of same a fortified plant based composition, wherein said fortified plant based composition is fortified with ferrous bisglycinate, said fortified plant based composition comprises components selected from the group consisting of apple, mango, lime, and turmeric, said fortified plant based composition is a beverage, and said fortified plant based composition has a pH in the range of 3.5-3.9.

15. A method according to claim 14, wherein the disorder link arises from omitting some or all animal products in the diet.

16. A method according to claim 14 for the treatment of disorders selected from anaemia, goitre, osteomalacia and osteoporosis.

17. A method comprising using ferrous bisglycinate to fortify a plant based composition at a pH in the range of 3.5-3.9 with iron while reducing unwanted metallic taste and/or a colour change in the composition.

18. (canceled)
